(19) United States
(12) Patent Application Publication
(10) Pub. No.: US 2026/0199100 A1
(43) Pub. Date: Jul. 16, 2026

(54) Spinal Implant

(71) Applicant: Additive Surgical Pty Ltd, Kent Town (AU)

(72) Inventor: Gibran Maher, Kent Town (AU)

(21) Appl. No.: 19/445,730

(22) Filed: Jan. 12, 2026

(30) Foreign Application Priority Data
Jan. 13, 2025 (AU) 2025900087

(51) Int. Cl. A61F 2/44; A61F 2/30

(52) U.S. Cl. CPC A61F 2/4455; A61F 2002/30011; A61F 2002/30014; A61F 2002/30784; A61F 2002/3092; A61F 2310/00023

(57) ABSTRACT

A spinal implant (100) configured for insertion between adjacent vertebral endplates, the spinal implant (100) including a first lattice structure (130) and a second lattice structure (135), wherein the first lattice structure (130) has a first modulus of elasticity and the second lattice structure (135) has a second modulus of elasticity, wherein the first modulus of elasticity is different to the second modulus of elasticity, wherein the first lattice structure (130) is distinct from the second lattice structure (135), and wherein the first lattice structure (130) is connected to the second lattice structure (135) so as to be in full porous communication throughout the spinal implant (100).

### CROSS-REFERENCE TO RELATED APPLICATIONS

This application claims priority to Australian Patent Application No. 2025900087, filed on 13 January 2025. The entire disclosure of Australian Patent Application No. 2025900087 is hereby incorporated by reference in its entirety and for all purposes.

### FIELD

The present disclosure generally relates to orthopaedic implants, and more particularly, but not exclusively, to spinal implants for treating spine and/or disc disorders and/or injuries.

The invention has been developed primarily in relation to implants for treating spine and/or disc disorders and/or injuries, and will be described hereinafter with reference to these applications. However, it will be appreciated that the invention is not limited to this particular field of use, and may also be employed in other orthopaedic applications.

### BACKGROUND

Spine disorders and injuries represent a significant social and economic burden on healthcare systems, encompassing a wide variety of pathological conditions such as degenerative disc disease, spondylolisthesis, disc herniation, spinal stenosis, and fractures. Spine conditions may restrict movement, increase spine instability, and present with chronic pain, impairing a patient’s ability to complete daily tasks, and significantly decreasing their quality of life. However, spine surgery is known to be one of the costlier procedures within the surgical realm.

Spinal interbody fusion surgery has emerged as a key intervention strategy and aims to at least restore disc height and spine stability, alleviate pain, and enhance patient mobility. Spinal fusion is the complete union (fusion) of two adjacent vertebral endplates by removing and replacing the affected intervertebral disc with a spine cage made of biocompatible materials such as a metal (e.g. titanium) or plastic (Polyetheretherketone – PEEK). Complete fusion of two vertebrae typically takes 6 to 12 months and within this time, the cage must not move (migrate). The success of spinal fusion is greatly influenced by the design of the implemented spine cage.

However, current spine cages have limitations in their design. Spine cages of the current industry standard, for example, typically have low porosity and isometric designs, which may not sufficiently provide for continuous bone formation or bone growth throughout.

### SUMMARY

It is an object of the present invention to substantially overcome, or at least ameliorate, one or more of the disadvantages of existing arrangements, or at least provide a useful alternative to existing arrangements.

There is disclosed herein a spinal implant configured for insertion between adjacent vertebral endplates, the spinal implant including a first lattice structure and a second lattice structure,

wherein the first lattice structure has a first modulus of elasticity and the second lattice structure has a second modulus of elasticity, wherein the first modulus of elasticity is different to the second modulus of elasticity,

wherein the first lattice structure is distinct from the second lattice structure, and

wherein the first lattice structure is connected to the second lattice structure so as to be in full porous communication throughout the spinal implant.

In one or more embodiments, the first lattice structure includes a first plurality of pores having a first range of pore sizes and the second lattice structure includes a second plurality of pores having a second range of pore sizes, with the first range of pore sizes being different to the second range of pore sizes.

In one or more embodiments, the first plurality of pores are in full communication with the second plurality of pores such that the spinal implant has a continuous porosity running therethrough. In such embodiments, the first plurality of pores are entirely connected to the second plurality of pores.

In one or more embodiments, the first lattice structure has a first thickness and the second lattice structure has a second thickness, with the first thickness being different to the second thickness.

In one or more embodiments, the first lattice structure forms at least one endplate of the spinal implant. In one or more embodiments, the first lattice structure forms a pair of endplates of the spinal implant.

In one or more embodiments, the at least one endplate has a surface that includes a plurality of projections. In some embodiments, the at least one endplate may have an outer surface including a plurality of teeth. In some embodiments, the plurality of projections are curved.

In one or more embodiments, the second lattice structure forms an implant body of the spinal implant.

In other embodiments, the first lattice structure is incorporated into at least one endplate of the spinal implant, and the second lattice structure is incorporated into an implant body of the spinal implant.

In one or more embodiments, the first and second lattice structures are in full continuous communication such that a porous network extends entirely through the at least one endplate and the implant body.

In one or more embodiments, the first lattice structure is an isometric lattice structure having perforations. In such embodiments, the isometric lattice structure has a repeating unit cell pattern.

In one or more embodiments, the second lattice structure is a biomimetic lattice structure. In such embodiments, the biomimetic lattice structure has a randomised cell pattern.

In one or more embodiments, the spinal implant is formed from three-dimensional-printed porous titanium (3Dp-Ti) material.

There is also disclosed herein a spinal implant configured for insertion between adjacent vertebral endplates, the spinal implant including:

an implant body defining a first lattice structure;

at least one endplate adjacent the implant body and defining a a second lattice structure;

wherein the first lattice structure has a first modulus of elasticity and the second lattice structure has a second modulus of elasticity, wherein the first modulus of elasticity is different to the second modulus of elasticity,

wherein the first lattice structure is distinct from the second lattice structure, and

wherein the first lattice structure is connected to the second lattice structure so as to be in full porous communication throughout the spinal implant.

In one or more embodiments, the first lattice structure includes a first plurality of pores having a first range of pore sizes and the second lattice structure includes a second plurality of pores having a second range of pore sizes, and wherein the first plurality of pores are in full communication with the second plurality of pores such that the spinal implant has a continuous porosity running therethrough.

### BRIEF DESCRIPTION OF THE DRAWINGS

For a more complete understanding of the present invention, exemplary embodiments of the invention are explained in more detail in the following description with reference to the accompanying drawing figures, in which like reference signs designate like parts and in which:

FIG. 1 is a schematic isometric view of a cervical implant according to one embodiment;

FIG. 2 is a schematic top view of the cervical implant shown in FIG. 1;

FIG. 3 is a schematic front view of the cervical implant shown in FIG. 1;

FIGS. 4 is a schematic side view of the cervical implant shown in FIG. 1;

FIG. 5 is a schematic isometric view of an example of a cervical implant, with two distinct lattice structures shown;

FIG. 6 is a schematic isometric view of an example of an implant body of a cervical implant;

FIG. 7 is a schematic isometric view of an example of a first lattice structure of a cervical implant;

FIG. 8 is a schematic isometric view of an example of a second lattice structure of a cervical implant;

FIG. 9 is a further schematic isometric view of an example of a cervical implant;

FIG. 10 is a further schematic front view of an example of a cervical implant;

FIG. 11 is a further schematic side view of an example of a cervical implant;

FIGS. 12 to 16 are schematic isometric views of examples of lumbar implants; and

FIGS. 17 to 20 are schematic views of examples of lattice structures.

### DETAILED DESCRIPTION

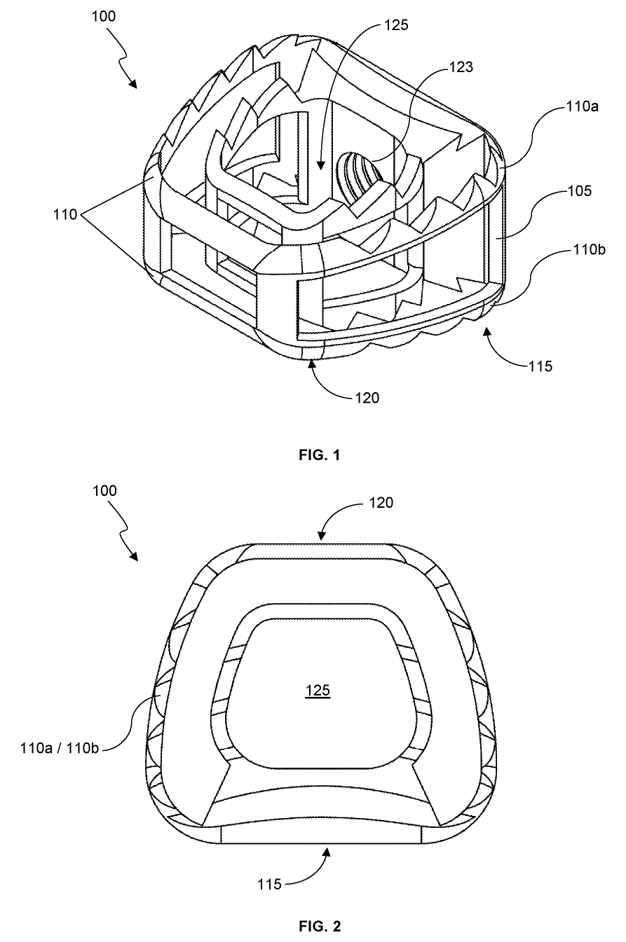

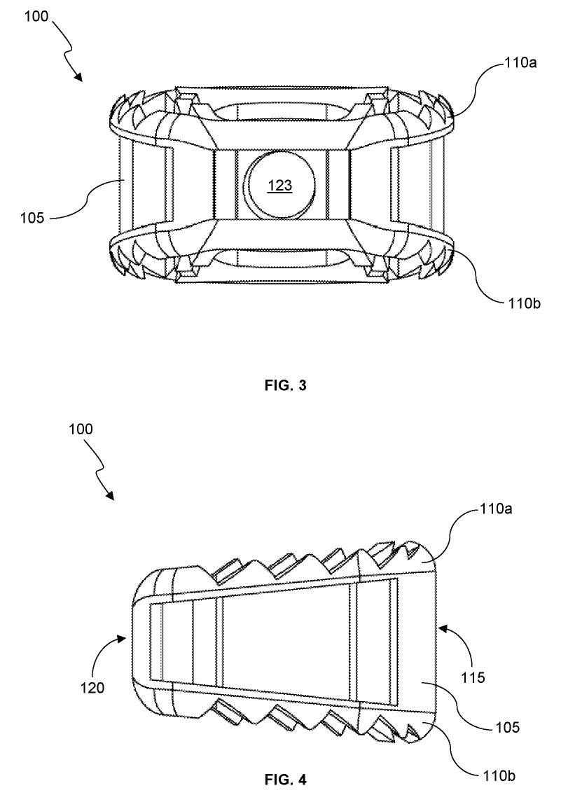

In FIGS. 1 to 4 of the accompanying drawings, there is schematically depicted an embodiment of a spinal implant 100. For the purposes of this specification, the spinal implant 100 and various alternative embodiments will be described for use in treating spine and/or disc disorders and/or injuries, such as degenerative disc disease, spondylolisthesis, disc herniation, spinal stenosis, and fractures. It will, however, be appreciated that the spinal implant 100 and various alternative embodiments may be adapted for treating bone disorders and/or injuries in other areas of the body. For such other applications, the spinal implant 100 may simply be referred to as an orthapaedic implant.

Throughout this specification, the spinal implant 100 may also be used as a general term to describe an Anterior Cervical Interbody Fusion (ACIF) implant, an Anterior Lumbar Interbody Fusion (ALIF) implant, a Posterior Lumbar Interbody Fusion (PLIF) implant, a Lateral Lumbar Interbody Fusion (LLIF) implant, a Transforaminal Lumbar Interbody Fusion (TLIF) implant, or an Oblique implant. It will, however, be understood that the embodiment of the spinal implant 100 shown in FIGS. 1 to 4 is representative of an ACIF implant, or simply a Cervical implant. Other variations/uses of the spinal implant 100 will be described in further detail below.

The spinal implant 100 is configured for insertion between adjacent vertebral endplates of a spine. The spinal implant 100 may be sized, shaped, and configured to replace an intervertebral disc of the spine so as to provide a complete spinal fusion of the adjacent vertebral endplates. The spinal implant 100, including one or more of components thereof, may be constructed by 3D printing of porous titanium (3Dp-Ti) or titanium alloy material so as to form a dual-lattice structure whereby the lattices are distinct from one another, as will be described in further detail below. It will, however, be appreciated that the spinal implant 100 may be constructed using any other suitable method and from any other suitable material, depending on the design requirements of the spinal implant 100.

In the depicted embodiment, the spinal implant 100 includes an implant body 105 and at least one endplate 110 adjacent the implant body. In some embodiments, the spinal implant 100 includes a pair of endplates 110 adjacent the implant body. In the example shown, the pair of endplates 110 includes a first endplate 110a and a second endplate 110b. The implant body 105 and the pair of endplates 110 may each have a trapezoidal shape with rounded corners. Each endplate 110a, 110b may be sized and shaped to correspond to the size and shape of the implant body 105. The implant body 105 and the pair of endplates 110 may be integrally formed. The implant body 105 and the pair of endplates 110 may form a frame or cage of the spinal implant 100.

The spinal implant 100 may have a first end portion 115 and a second end portion 120. In the embodiment shown, the implant body 105 has a thickness that gradually decreases as it extends along its length from the first end portion 115 to the second end portion 120. In other words, the implant body 105 at the first end portion 115 may have a thickness that is larger than a thickness of the implant body 105 at the second end portion 120.

The implant body 105 at the first end portion 115 may include one or more openings 123 for interfacing with surgical tools and enabling placement or insertion of the spinal implant 100 between adjacent vertebral endplates. The one more openings 123 may be threaded or otherwise shaped and/or sized to correspond to a surgical tool.

In the embodiment shown, the implant body 105 also has a width that gradually decreases as it extends along its length from the first end portion 115 to the second end portion 120. In other words, the implant body 105 at the first end portion 115 may have a width that is greater than a width of the implant body 105 at the second end portion 120.

The implant body 105 may have a length of between approximately 14 and 18 mm, a width of between approximately 12 and 16 mm, and a thickness or height of between approximately 5 and 12 mm. The thickness or height may be varied in increments of approximately 1 mm, whilst the length, width, and angle may be varied according to the required size of the implant 100. For example, a small implant may have a length of approximately 14 mm and a width of approximately 12 mm, a medium implant may have a length of approximately 16 mm and a width of 14 approximately mm, and a large implant may have a length of approximately 18 mm and a width of 16 approximately mm. It will, however, be appreciated that the dimensions of the implant body 105 are not necessarily limited to the dimensions described above, and may be adjusted depending on the design requirements of the spinal implant 100

Each endplate 110a, 110b may likewise have a width that gradually decreases as it extends along its length from the first end portion 115 to the second end portion 120. In other words, the endplate 110a, 110b at the first end portion 115 has a width that is larger than a width of the endplate 110a, 110b at the second end portion 120. Each endplate 110 may have a length, width, and thickness that corresponds to the dimensions of the implant body 105 mentioned above. The angle between the pair of endplates 110 may be between 0° and 15°. One or both of the endplates 110a, 110b may have a surface that includes a plurality of projections to facilitate attachment with the adjacent vertebral endplate. In the embodiment shown, each endplate 110a, 110b has an outer surface including a plurality of teeth.

In the embodiment shown, the spinal implant 100 includes a central window or aperture 125 that extends through the implant body 105 and the pair of endplates 110. This central window or aperture 125 may also be referred to as an internal graft window. The window 125 may have a trapezoidal shape with rounded corners. It will, however, be appreciated that the shape/or configuration of the window 125 is not necessarily limited to the shape and/or configuration as shown in the drawings or described above, and may be adjusted depending on the design requirements of the spinal implant 100. Similar to the shape of the implant body 105 and the endplates 110 as described above, the window 125 may also have a width that gradually decreases as it extends along its length from the first end portion 115 to the second end portion 120. In other words, the window 125 at the first end portion 115 may have a width that is larger than a width of the window 125 at the second end portion 120.

It will be understood that the shape and/or configuration and dimensions of the overall spinal implant 100 and components thereof are not necessarily limited to the shape and/or configuration or dimensions as shown in the drawings or described above, and may be adjusted depending on the design requirements of the spinal implant 100. For example, where the spinal implant 100 is used as an Anterior Lumbar Interbody Fusion (ALIF) implant, a Posterior Lumbar Interbody Fusion (PLIF) implant, a Lateral Lumbar Interbody Fusion (LLIF) implant, a Transforaminal Lumbar Interbody Fusion (TLIF), or an Oblique implant, the shape and/or configuration and dimensions may vary accordingly. Examples of these other implant types are shown in FIGS. 12 to 16.

In embodiments where the spinal implant 100 is used as an ALIF implant, and by way of example, the implant body 105 may have a length of between approximately 34 and 42 mm, a width of between approximately 26 and 34 mm, and an anterior height of between approximately 10 and 18 mm in 2 mm increments. The angle between the pair of endplates 110 may be between 10° and 25°.

In embodiments where the spinal implant 100 is used as a PLIF implant, and by way of example, the implant body 105 may have a length of between approximately 22 and 30 mm, a width of between approximately 9 and 11 mm, and an anterior height of between approximately 7 and 16 mm in 1 mm increments. The angle between the pair of endplates 110 may be between 0° and 20°.

In embodiments where the spinal implant 100 is used as an LLIF implant, and by way of example, the implant body 105 may have a length of between approximately 40 and 60 mm in 5 mm increments, a width of between approximately 20 and 25 mm, and an anterior height of between approximately 8 and 18 mm. The angle between the pair of endplates 110 may between 0° and 25°.

In embodiments where the spinal implant 100 is used as a TLIF implant, and by way of example, the implant body 105 may have a length of between approximately 26 and 34 mm, a width of between approximately 9 and 10 mm, and an anterior height of between approximately 7 and 15 mm in 1 mm increments. The angle between the pair of endplates 110 may between 0° and 20°.

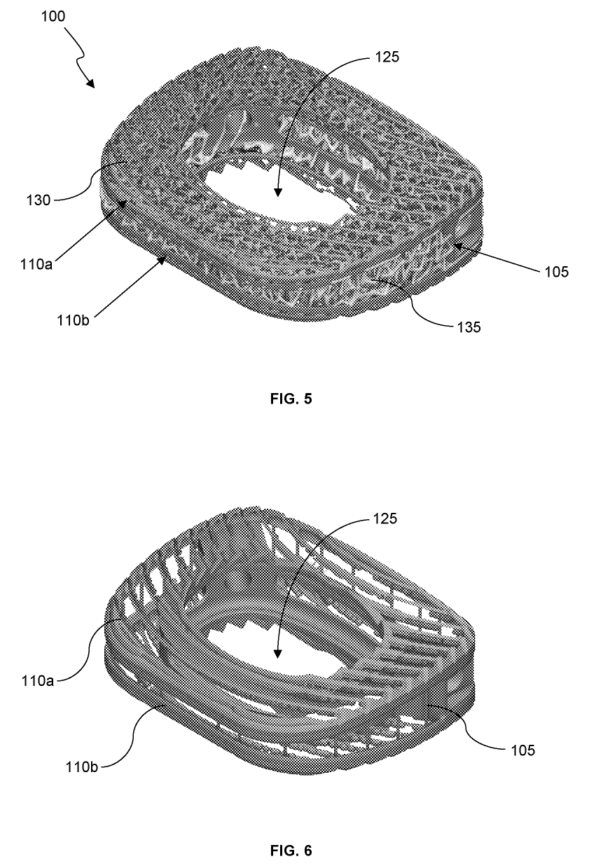

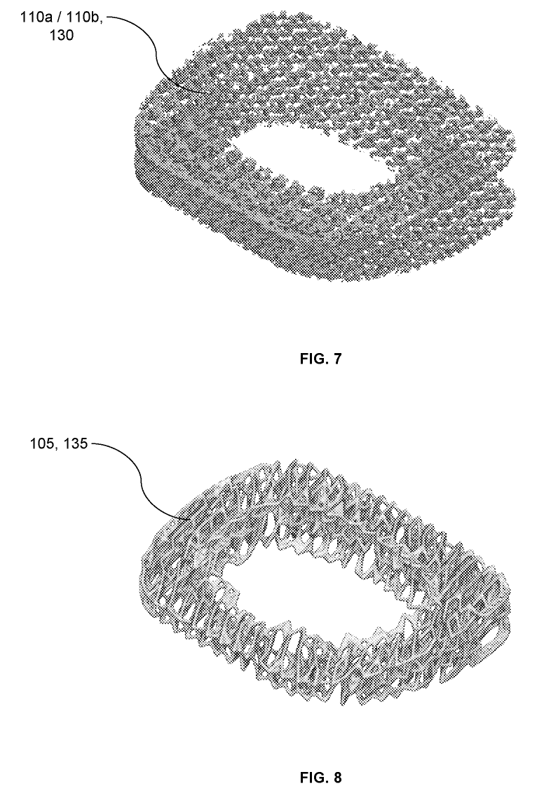

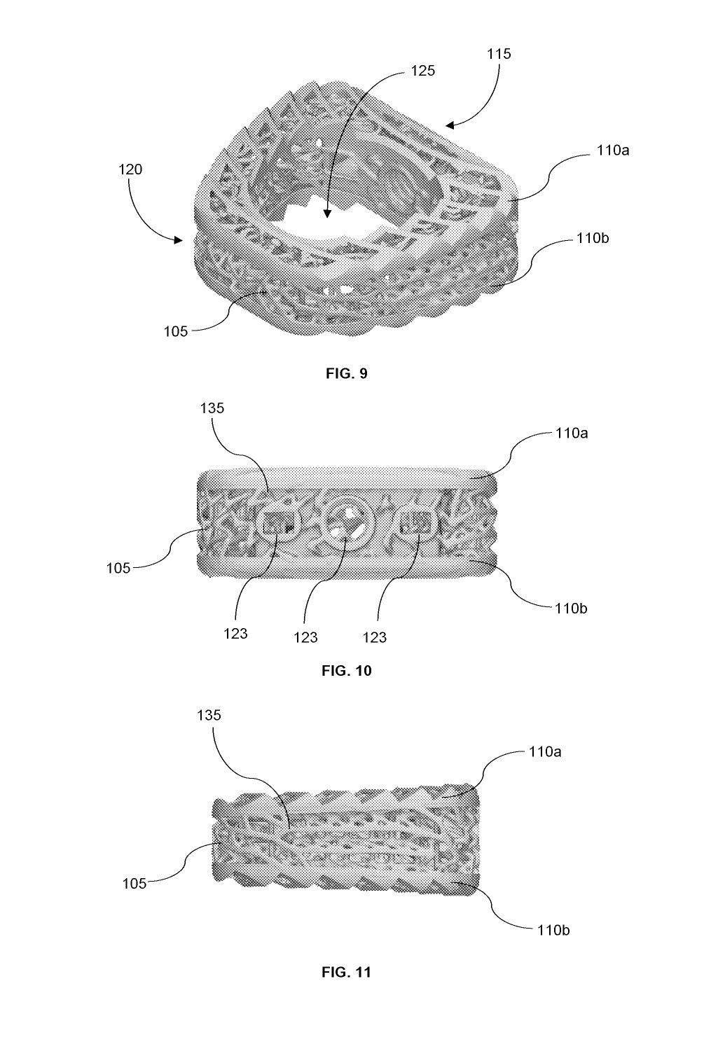

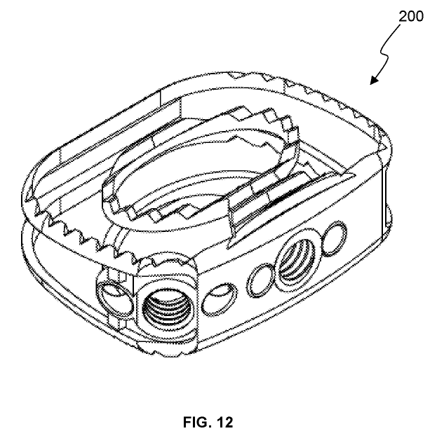

As best shown in FIGS. 5 to 8, the spinal implant 100 includes a first lattice structure or framework 130 and a second lattice structure or framework 135. The first lattice structure 130 may form part of, define, or otherwise be incorporated into the first and/or second endplates 110a, 110b. The second lattice structure 135 may form part of, define, or otherwise be incorporated into the implant body 105. The first lattice structure 130 is distinct from the second lattice structure 135 (in respect of their physical structures and/or material properties) so as to provide two distinct lattice structures within the same implant, whilst still being in full porous communication with one another as will be discussed in further detail below.

In some embodiments, the lattices of each lattice structure 130, 135 are three-dimensional arrangements of points in a unit cell creating a periodic structure. The lattices may have different ratios of empty space to struts (structural element of material) depending on their structure. A lattice with thinner struts and more empty space will result in higher porosity, which may be beneficial for osteointegration and capillarisation. Conversely, thicker struts and less empty space will have lower porosity and higher stiffness (more dense), which may have surface cell adhesion benefits. It is envisaged that forming the spinal implant 100 from 3Dp-Ti cages, for example, may at least allow for more complex (more organic), stress-optimised structures that mimic bone (biomimicry).

In some embodiments, the first lattice structure 130 has a first modulus of elasticity and the second lattice structure 135 has a second modulus of elasticity, with the first modulus of elasticity being different to the second modulus of elasticity. As the first lattice structure 130 is distinct from the second lattice structure 135 whilst still being in full porous communication with one another, it will be appreciated that the implant 100 may be provided with two different moduli of elasticity between two distinct lattice structures of the same implant, rather than merely having gradients of moduli within a single surface of an implant, by way of comparison with known spine cages.

It is envisaged that the first lattice structure 130 and the second lattice structure 135 may each have a modulus of elasticity that is anywhere within the modulus of elasticity range of 3Dp-Ti (i.e. between approximately 0.8 to 18 GPa), for example, as long as they are different from one another. In some embodiments, the first modulus of elasticity may be higher than the second modulus of elasticity. In other embodiments, the second modulus of elasticity may be higher than the first modulus of elasticity. The skilled person will appreciate that the modulus of elasticity may be varied depending on the material used and the design requirements of the implant 100.

In some embodiments, and as will be discussed in further detail below, the first lattice structure 130 is in full or complete porous communication with the second lattice structure 135. In a preferred form, the first lattice structure 130 is in full or complete porous communication with the second lattice structure 135 throughout the spinal implant 100. In other words, the first lattice structure 130 may be connected to the second lattice structure 135 so as to allow for continuous porosity therethrough, without any structure interrupting connection between pores of the first lattice structure 130 and pores of the second lattice structure 135. This arrangement may at least enable continuous bone formation / bone growth throughout the implant 100.

It will be appreciated that the provision of two distinct lattice structures (i.e. the first lattice structure 130 and the second lattice structure 135) that fully communicate, and with continuous porosity running therethrough (i.e. without any structure interrupting connection between the pores of the first lattice structure 130 and the pores of the second lattice structure 135), may at least allow for an unmet clinical need to be realised. This is in contrast with known spine cages, which typically include a plinth or thick struts that extend therethrough to interrupt the porous communication of the implant, thus failing to allow for continuous bone formation / bone growth. Such known spine cages also do not provide two distinct lattice structures having two different moduli of elasticity, let alone two distinct lattice structures in the same implant and that fully communicate to allow for continuous porosity running therethrough.

The first lattice structure 130 may include a first plurality of pores having a first range of pore sizes and the second lattice structure 135 may include a second plurality of pores having a second range of pore sizes, with the first range of pore sizes being different to the second range of pore sizes. In some arrangements, the range of pore sizes of first plurality of pores is smaller than the range of pore sizes of second plurality of pores. In other arrangements, the range of pore sizes of the second plurality of pores is smaller than the range of pore sizes of the first plurality of pores. Each lattice structure 130, 135 may have a gradient in pore sizes.

It will be appreciated that larger pore sizes may at least enhance the optimisation of load transfers from the implant 100 to inferior vertebrae and may also reduce the risk of stress shielding. Radiographically, larger pore sizes may also at least allow for improved visualisation of bone growth, creating a clearer view to determine the status of fusion. It will be further appreciated that smaller pores may at least increase surface cell adhesion due to the increase in surface area. Increased cell adhesion may at least enhance initial fixation, reducing the risk of implant micromotion and non-fusion. It is understood that the pore size optimal for facilitation of surface bone attachment may be between 50 to 400 μm. Larger pore sizes may also have fusion benefits with new bone formation and capillarisation observed in implants with pore sizes greater than 300 μm, leading to direct osteogenesis. Whilst it has generally been considered that a pore size in the range of 100 to 1,000 μm is optimal for bone tissue engineering, it is understood that a gradient in pore sizes may be beneficial for both initial surface fixation and overall percentage of fusion for the implant 100.

With the above in mind, it is envisaged that in some embodiments, the first plurality of pores and the second plurality of pores may each have pore sizes ranging from between approximately 50 to 2,650 µm, and may have a gradient in pore sizes. In some embodiments, the first plurality of pores may have a pore size ranging from between approximately 100 to 400 µm with between approximately 70 to 90 % porosity, whilst the second plurality of pores may have a pore size ranging from between approximately 400 to 1,000 µm with between approximately 70 to 90% porosity. As mentioned above, the first and second plurality of pores may have gradient in pore sizes, and the first and second plurality of pores may also have a gradient in porosities.

As discussed above in relation to the first and second lattice structures 130 and 135 in and of themselves, the first plurality of pores of the first lattice structure 130 may also be in communication with the second plurality of pores of the second lattice structure 135. In a preferred form, the first plurality of pores are in full or complete communication with the second plurality of pores throughout the spinal implant 100, i.e. to provide for continuous porosity throughout the spinal implant 100. In other words, the first plurality of pores may be connected to the second plurality of pores so as to allow for continuous porosity between the first and second lattice structures 130, 135, thereby allowing for continuous bone formation / bone growth.

The first lattice structure 130 may have a first thickness and the second lattice structure 135 may have a second thickness, with the first thickness being different to the second thickness.

As noted above, the first lattice structure 130 may form or define at least one endplate 110a, 11b of the spinal implant 100, and the second lattice structure 135 may form or define the implant body 105 of the spinal implant 100. The first lattice structure 130 may alternatively be incorporated or integrated into at least one endplate 110a, 110b of the spinal implant 100, and the second lattice structure 135 may alternatively be incorporated or integrated into the implant body 105 of the spinal implant 100. In some embodiments, the first and second lattice structures 130, 135 are in full continuous communications such that a porous network of the spinal implant 100 extends entirely through the endplates 110a, 110b and the implant body 105.

In some embodiments, the first lattice structure 130 may be an isometric lattice structure having perforations. In such arrangements, the isometric lattice structure may have a repeating unit cell pattern. The isometric lattice structure may also be referred to as a perforated triply periodic minimal surface (TPMS). It is envisaged that by having a perforated TPMS as the first lattice structure 130 forming at least one (and preferably both) endplate 110a, 110b, the implant 100 may have a contact surface that is mechanically similar to cortical bones.

In some embodiments, the second lattice structure 135 may be a biomimetic lattice structure. In such arrangements, the biomimetic lattice structure may have a randomised cell pattern. The biomimetic lattice structure may also be referred to as Orthofoam. It is envisaged that by having Orthofoam as the second lattice structure 135 forming the implant body 105, the implant 100 may have an internal structure that is mechanically similar to cancellous bone.

It will be appreciated that with the combination of two distinct lattice structures having continuous porosity, the first (isometric) lattice structure 130 may at least allow for integration with cortical bone, whilst the second (biomimetic) lattice structure 135 may at least allow for integration with cancellous bone. This arrangement may at least address issues with suboptimal bone growth / bone formation with conventional known spine cages, which typically rely upon the same irregular structure throughout (or include structures that physically interrupt the porous communication of the implant) and thus do not adequately provide for continuous bone growth / bone formation.

In one or more embodiments, the projections (i.e. the teeth) on the surface of one or both of the endplates 110a, 110b have a curved shape. It is envisaged that the shape of the curve may be different for different variations/uses of the spinal implants. In the depicted embodiment, this curve has been modelled from the mandible of a shark and may be used to prevent lateral and anterior migration of the spinal implant 100. It will be appreciated that this curve may be ideal for balancing ease of insertion and strong initial contact with the endplates 110a, 110b. The arrangement of the curved teeth may at least assist in primary stability and as noted above, may at least prevent sagittal and anterior migration of the implant in the intervertebral space.

In this embodiment of the spinal implant 100, the central window 125 (i.e. the internal graft window) may have an internal lip or overhang which creates additional contact surface area for the implant 100 whilst still maintaining the largest graft window volume possible. This internal lip or overhang may be present in any of the variations/uses of the spinal implants described above, and more particularly on the ALIF, Cervical, and Lateral implants.

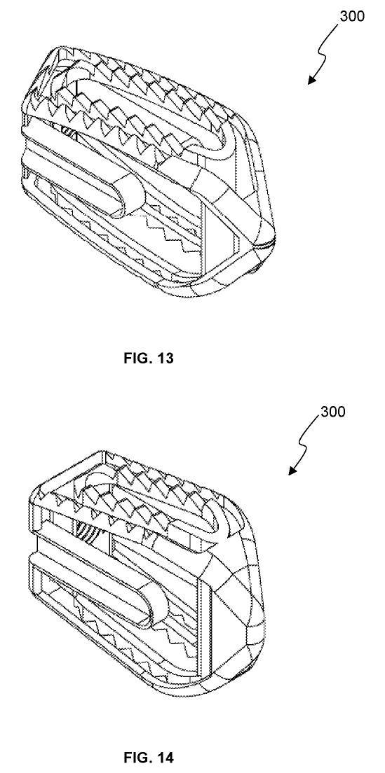

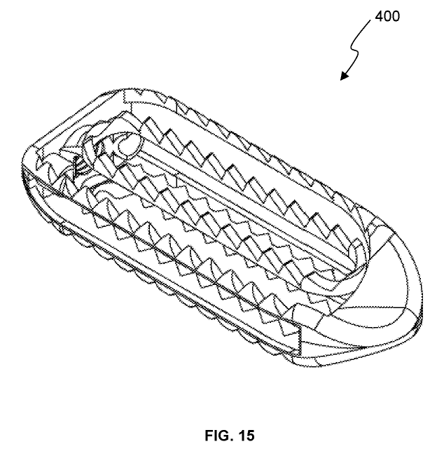

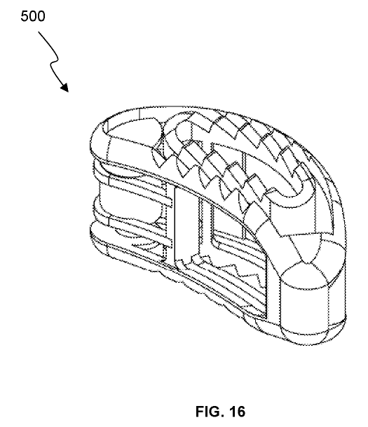

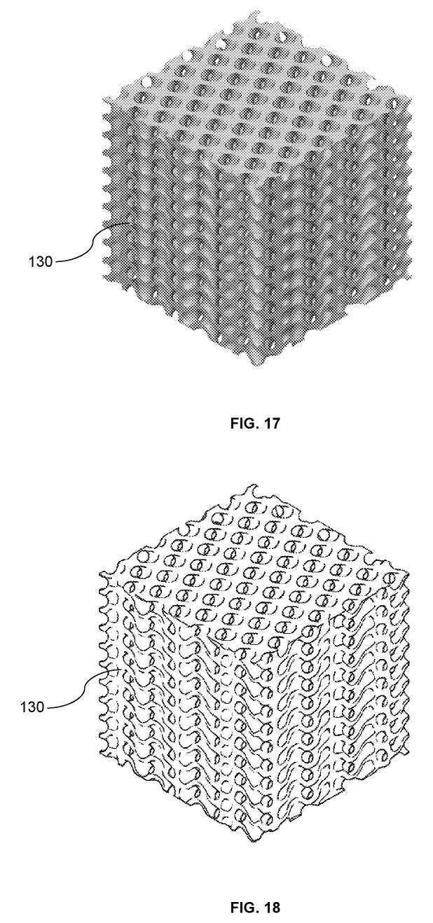

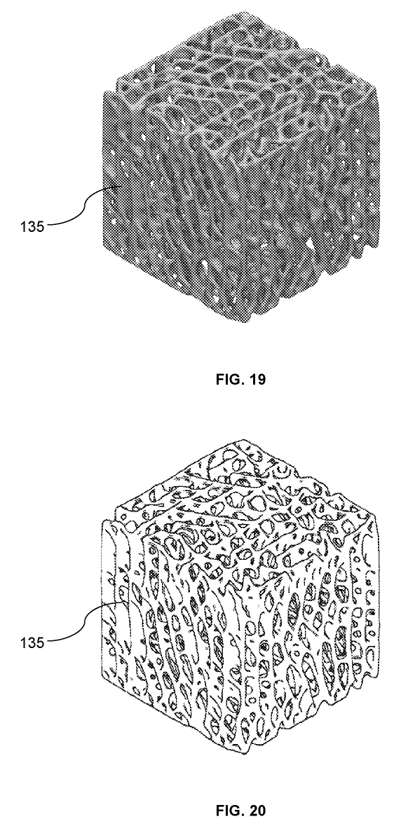

FIGS. 12 to 16 show various examples of an ALIF implant 200 (FIG. 12), a PLIF implant 300 (FIGS. 13 and 14), an LLIF implant 400 (FIG. 15), and a TLIF implant 500 (FIG. 16). It will be understood that these exemplary implants 200, 300, 400, and 500 each function in a generally similar manner to the implant 100 described above, and any one or more of the like features or functions of the embodiment of the implant 100 described above are applicable to the embodiment of the implants 200, 300, 400, and 500, and vice versa. Additionally, FIGS. 17 to 20 show various examples of the lattice structures 130, 135 of the various embodiments of the spinal implants described above.

Various forms of the spinal implant described above may have one or more of the following advantages. It will be appreciated that the dual-lattice arrangement of the spinal implant may at least allow for distinct or unique lattice structures that each have a specifically designed role. The provision of two distinct lattice structures that fully communicate, and with continuous porosity running therethrough (i.e. without any structure interrupting connection between the pores of the two lattice structures, may at least allow for an unmet clinical need of continuous bone formation / bone growth throughout the implant to be realised. As discussed above, each lattice structure may have a different modulus of elasticity, different pore sizes, and/or different beam thicknesses. The arrangement of the distinct or unique lattice structures may at least allow the implant to match the porosities and mechanical characteristics of human cortical and cancellous vertebral bone, for example. The dual-lattice arrangement may at least combine and leverage the strengths of two different lattice structures to provide for enhanced mechanical performance over individual lattices whilst optimising bone ingrowth, for example, by enhancing stress distribution, energy absorption, and promoting biological fusion.

Although specific embodiments of the invention are illustrated and described herein, it will be appreciated by those of ordinary skill in the art that a variety of alternative and/or equivalent implementations exist. It should be appreciated that the exemplary embodiment or exemplary embodiments are examples only and are not intended to limit the scope, applicability, or configuration in any way. Rather, the foregoing summary and detailed description will provide those skilled in the art with a convenient road map for implementing at least one exemplary embodiment, it being understood that various changes may be made in the function and arrangement of elements described in an exemplary embodiment without departing from the scope as set forth in the appended claims and their legal equivalents. Generally, this application is intended to cover any adaptations or variations of the specific embodiments discussed herein.

It will also be appreciated that in this document the terms “comprise”, “comprising”, “include”, “including”, “contain”, “containing”, “have”, “having”, and any variations thereof, are intended to be understood in an inclusive (i.e. non-exclusive) sense, such that the process, method, device, apparatus or system described herein is not limited to those features or parts or elements or steps recited but may include other elements, features, parts or steps not expressly listed or inherent to such process, method, article, or apparatus. Furthermore, the terms “a” and “an” used herein are intended to be understood as meaning one or more unless explicitly stated otherwise. Moreover, the terms “first”, “second”, etc. are used merely as labels, and are not intended to impose numerical requirements on or to establish a certain ranking of importance of their objects.

1. A spinal implant configured for insertion between adjacent vertebral endplates, the spinal implant including a first lattice structure and a second lattice structure,
wherein the first lattice structure has a first modulus of elasticity and the second lattice structure has a second modulus of elasticity, wherein the first modulus of elasticity is different to the second modulus of elasticity,
wherein the first lattice structure is distinct from the second lattice structure, and
wherein the first lattice structure is connected to the second lattice structure so as to be in full porous communication throughout the spinal implant.

2. The spinal implant of claim 1, wherein the first lattice structure includes a first plurality of pores having a first range of pore sizes and the second lattice structure includes a second plurality of pores having a second range of pore sizes, with the first range of pore sizes being different to the second range of pore sizes.

3. The spinal implant of claim 2, wherein the first plurality of pores are in full communication with the second plurality of pores such that the spinal implant has a continuous porosity running therethrough.

4. The spinal implant of claim 3, wherein the first plurality of pores are entirely connected to the second plurality of pores.

5. The spinal implant of claim 1, wherein the first lattice structure has a first thickness and the second lattice structure has a second thickness, with the first thickness being different to the second thickness.

6. The spinal implant of claim 1, wherein the first lattice structure forms at least one endplate of the spinal implant.

7. The spinal implant of claim 6, wherein the first lattice structure forms a pair of endplates of the spinal implant.

8. The spinal implant of claim 6, wherein the at least one endplate has a surface that includes a plurality of projections.

9. The spinal implant of claim 8, wherein the at least one endplate has an outer surface including a plurality of teeth.

10. The spinal implant of claim 8, wherein the plurality of projections are curved.

11. The spinal implant of claim 1, wherein the second lattice structure forms an implant body of the spinal implant.

12. The spinal implant of claim 1, wherein the first lattice structure is incorporated into at least one endplate of the spinal implant, and the second lattice structure is incorporated into an implant body of the spinal implant.

13. The spinal implant of claim 6, wherein the first and second lattice structures are in full continuous communication such that a porous network extends entirely through the at least one endplate and the implant body.

14. The spinal implant of claim 1, wherein the first lattice structure is an isometric lattice structure having perforations.

15. The spinal implant of claim 14, wherein the isometric lattice structure has a repeating unit cell pattern.

16. The spinal implant of claim 1, wherein the second lattice structure is a biomimetic lattice structure.

17. The spinal implant of claim 16, wherein the biomimetic lattice structure has a randomised cell pattern.

18. The spinal implant of claim 1, the spinal implant is formed from three-dimensional-printed porous titanium (3Dp-Ti) material.

19. A spinal implant configured for insertion between adjacent vertebral endplates, the spinal implant including:
an implant body defining a first lattice structure;
at least one endplate adjacent the implant body and defining a a second lattice structure;
wherein the first lattice structure has a first modulus of elasticity and the second lattice structure has a second modulus of elasticity, wherein the first modulus of elasticity is different to the second modulus of elasticity,
wherein the first lattice structure is distinct from the second lattice structure, and
wherein the first lattice structure is connected to the second lattice structure so as to be in full porous communication throughout the spinal implant.

20. The spinal implant of claim 19, wherein the first lattice structure includes a first plurality of pores having a first range of pore sizes and the second lattice structure includes a second plurality of pores having a second range of pore sizes, and wherein the first plurality of pores are in full communication with the second plurality of pores such that the spinal implant has a continuous porosity running therethrough.
